(19) United States
(12) Patent Application Publication
(10) Pub. No.: US 2026/0177837 A1
(43) Pub. Date: Jun. 25, 2026

(54) OCULOGRAPHY METHOD, OCULOGRAPHY DEVICE, PAIR OF AR OR VR SMART GLASSES HAVING THE OCULOGRAPHY DEVICE, AND CONTROL UNIT

(71) Applicant: Robert Bosch GmbH, Stuttgart (DE)

(72) Inventors: Johannes Meyer, Haseluenne (DE); Thomas Alexander Schlebusch, Renningen (DE)

(21) Appl. No.: 19/132,405

(22) PCT Filed: Mar. 15, 2024
(86) PCT No.: PCT/EP2024/056897

§ 371 Date: May 23, 2025

(30) Foreign Application Priority Data
May 19, 2023 (DE) 10 2023 204 685.7

(51) Int. Cl. G02B 27/01; G01B 11/00; G06F 3/01

(52) U.S. Cl. CPC G02B 27/0179; G01B 11/002; G02B 2027/0187; G06F 3/013

(57) ABSTRACT

An oculography method, in particular a smart-glasses oculography method for pairs of augmented reality smart glasses and/or for pairs of virtual reality (VR) smart glasses/VR headsets. The method includes at least one oculography step in which at least one eye movement of an eye, in particular an eye of a smart-glasses user, is ascertained, in particular tracked, by a subset of laser feedback interferometry sensors of an LFI sensor array which is associated with the eye. In at least one selection step, which precedes the oculography step in time, a selection of at least one LFI sensor provided for performing the oculography step, which follows in time, is made based on a parameter comparison of test measurements of multiple LFI Sensors of the LFI sensor array.

### BACKGROUND INFORMATION

An oculography method, in particular a smart-glasses oculography method, having at least one oculography step in which at least one eye movement of an eye, in particular an eye of a smart-glasses user, is ascertained, in particular tracked, by a subset of laser feedback interferometry (LFI) sensors of an LFI sensor array which is associated with the eye, is available in the related art. For practical operation of pairs of smart glasses with a corresponding oculography method, it is essential that the laser beams of the LFI sensor also hit the eye of a user, namely at an angle that is not perpendicular to a surface velocity of the eyeball. Different people have very different physiologies. Due to individually different eye distances, individually different head and nose geometries or simply due to sliding of the pair of smart glasses, the point of incidence of the laser beams on the eye may vary. Therefore, it will generally not be possible to find a single laser arrangement that is equally suitable for all users.

### SUMMARY

An example embodiment of the present invention proceeds from an oculography method, in particular a smart-glasses oculography method, e.g., for pairs of augmented reality (AR) smart glasses and/or for pairs of virtual reality (VR) smart glasses, having at least one oculography step in which at least one eye movement of an eye, in particular an eye of a smart-glasses user, is ascertained, in particular tracked, by a subset of laser feedback interferometry (LFI) sensors of an LFI sensor array which is associated with the eye, preferably by all LFI sensors of the LFI sensor array which is associated with the eye, in particular of a pair of smart glasses.

According to an example embodiment of the present invention, it is provided that, in at least one selection step, which in particular precedes the oculography step in time, at least one LFI sensor provided for performing the oculography step, which in particular follows in time, is selected on the basis of a parameter comparison of test measurements of multiple, preferably all, LFI sensors of the LFI sensor array. Thus, user friendliness and/or usability of pairs of smart glasses per se can advantageously be increased. Advantageously, an eye-box of a pair of smart glasses can be significantly enlarged, whereby the system advantageously remains capable of measurement even when a position of the pair of smart glasses, in particular of the LFI sensors of the pair of smart glasses, relative to the eye of the user changes. In particular, an oculography method is provided for representing, sensing and/or recording of eye movements. Preferably, the oculography method can be applied to various kinds and types of pairs of smart glasses, for example pairs of “see-through” AR smart glasses and/or pairs of pure VR smart glasses.

A “pair of smart glasses” is in particular to be understood as a wearable (head-mounted display), by means of which information can be added to the field of view of a user. Pairs of smart glasses preferably make augmented reality applications, virtual reality applications and/or mixed reality applications possible. Smart glasses are also commonly referred to as data glasses, VR glasses, or AR glasses. In particular, the optical display system is provided for generating and outputting virtual content or video content by means of a laser source, in particular a laser source different from the LFI sensor array. For example, the laser source could be arranged in a side region (for example, temple region) of the pair of smart glasses. The pair of smart glasses preferably comprises at least imaging optical units, such as a lens, in particular a pancake lens of a VR headset or a glasses lens of an AR headset. In particular, the LFI sensor array forms a part, in particular an integral part, of an eye tracking system of the pair of smart glasses. The eye tracking system could be provided for detecting an eye position and/or pupil position of the eye of the user by means of the so-called “dark pupil effect”, but preferably the eye tracking system is provided for detecting the eye position and/or pupil position by means of the so-called “bright pupil effect”, which is based in particular on the high infrared reflectivity of the retina of the eye. The terms “provided” and/or “configured” are in particular understood to mean specifically programmed, designed, and/or equipped. An object being provided and/or configured for a particular function is in particular understood to mean that the object fulfills and/or performs this particular function in at least one application state and/or operating state.

In particular, according to an example embodiment of the present invention, the LFI sensor array comprises multiple, preferably more than two, advantageously more than three, particularly advantageously more than four, preferably more than five and particularly preferably less than 10 individual LFI sensors, in particular per eye of the user. In particular, the LFI sensors of the LFI sensor array are statically arranged at least relative to the pair of smart glasses, in particular at least relative to a glasses lens of the pair of smart glasses. The LFI sensors may, for example, be arranged in a glasses frame of the pair of smart glasses, said glasses frame at least partially surrounding the glasses lens, or in one of the glasses temples of the pair of smart glasses. The LFI sensors preferably radiate their laser beams at different angles onto the eye/face of the user and/or onto different sub-regions of the eye/face of the user. The test measurements of the various LFI sensors of the LFI sensor array may be performed in succession or at least partially simultaneously (e.g., simultaneously in groups). However, in a partially simultaneous measurement with multiple LFI sensors, preferably always only so many LFI sensors are activated simultaneously that eye safety is always ensured in order to protect the eye of the user. A total laser intensity emitted simultaneously by the LFI sensor array thus always remains below a definable limit value, in particular always below a hazard threshold.

According to an example embodiment of the present invention, in order to perform the parameter comparison, in particular individual measurements of the multiple, preferably all, LFI sensors of the LFI sensor array are first performed in a measuring step. In particular, these individual measurements form the test measurements. These individual measurements are then preferably compared (parameter comparison) and, on the basis of specifiable criteria, which in particular can be read from the individual measurements, one or more of the LFI sensors are selected for the oculography subsequently performed. In particular, the method for ascertaining an eye position and/or pupil position on the basis of measurement data from LFI sensors is conventional.

The LFI sensors may be, for example, VCSELs, preferably Vip-VCSELs (“vertical-cavity surface-emitting lasers with integrated photo diode”). The LFI sensors of the pair of AR smart glasses may be integrated selectively either in the glasses frame, in the glasses lens, or in the glasses temple. The LFI sensors of a pair of VR smart glasses/a VR headset may, for example, be integrated in an imaging optical unit, e.g., in the edge region of the pancake lens. In addition, in the pair of VR smart glasses/the VR headset the LFI sensors may be arranged next to an image-generating display or, in the case of a partially transparent display, such as an OLED display, even behind the image-generating display. LFI sensors are based on an interferometric measurement method and are preferably capable of sensing a distance to a target (e.g., the eye of the smart glasses user) as well as a surface velocity of the target. In particular, the LFI sensor emits the laser beam in the infrared spectrum, and the laser beam then hits, at an angle y, a surface having a reflectivity R. From this surface, the light of the laser beam is then backscattered such that it enters a laser cavity of the LFI sensor again. In the laser cavity of the LFI sensor, the backscattered light interferes with a locally oscillating field of the LFI sensor. This leads in particular to modulation of laser power of the laser source, which may be sensed selectively either by a photodiode integrated into a rear reflector of the laser cavity or by a direct measurement of a voltage of the laser source. Furthermore, a current of the laser source may be modulated with a preferably triangular modulation signal in order to achieve a cyclic shift of the wavelength of the laser. If the laser parameters are known, a beat frequency and a Doppler frequency may subsequently be determined. From these quantities, the surface velocity of the eye, the distance of the LFI sensor from the eye and other eye parameters can be ascertained using conventional equations.

Furthermore, according to an example embodiment of the present invention, it is provided that, in the selection step, the selection of the at least one LFI sensor of the LFI sensor array provided for performing the oculography step is made on the basis of a parameter comparison of velocity test measurements, in particular eye movement velocity test measures, with the multiple LFI sensors of the LFI sensor array. Thus, high oculography sensitivity can advantageously be achieved. Advantageously, eye tracking can thereby be optimized, in particular made more precise. In particular, the LFI sensors do not already directly measure the eye movement velocity in the test measurements, but rather a “raw velocity”, from which the actual eye movement velocity could then be ascertained, e.g., for a method for ascertaining an eye position and/or pupil position, via further method steps.

According to an example embodiment of the present invention, when at least the LFI sensor having the highest velocity ascertained in the selection step is selected for performing the oculography step, high oculography sensitivity can advantageously be achieved. Advantageously, eye tracking can thereby be optimized, in particular made more precise. It is also possible that a group of LFI sensors of the LFI sensor array which have the highest velocities ascertained in the selection step are selected for performing the oculography step.

According to an example embodiment of the present invention, it is also provided that that the selection step is repeated if a time interval, in particular definable time interval, e.g., several seconds or several minutes, has passed, if it is ascertained that a minimum measurement data quality is not met when the oculography step is performed, and/or if a substantial change in an optical path length ascertained by the currently selected LFI sensor between LFI sensor and scattering surface, e.g., eye of the smart glasses user, is ascertained. Thus, user friendliness and/or usability of pairs of smart glasses per se can advantageously be increased. Advantageously, optimized eye tracking and/or pupil tracking can be achieved. In particular, after the selection step has been repeated, the previous selection of LFI sensors of the LFI sensor array for the oculography step is confirmed or adjusted/changed. The minimum measurement data quality may be measured, for example, via a signal/noise ratio. For example, it is determined that the minimum measurement data quality is not met in the performance of the oculography step if an amplitude of a beat frequency in an amplitude-frequency spectrum of the currently selected LFI sensor is below a definable limit value in comparison with a background intensity. The optical path length between the LFI sensor and the scattering surface/eye may be ascertained by frequency-modulating the currently selected LFI sensor. The corresponding method for this is conventional. By determining the optical path length, LFI sensors could also be excluded in the selection step for the current selection, e. g., on the basis of a thickness of an eyelid of the eye, which thickness allows a distinction with respect to a point of incidence of the laser beam on the eye or on the eyelid. In addition, a significant abrupt substantial increase in the optical path length indicates that a pupil of the eye has been hit, which may indicate a good current orientation of this sensor towards the eye.

According to an example embodiment of the present invention, also provided is an oculography device for performing the oculography method, comprising at least one LFI sensor array, in particular statically arranged LFI sensor array, which comprises multiple LFI sensors each configured to ascertain, in particular track, at least one eye movement of an eye, in particular an eye of the smart glasses user, wherein at least a majority of all LFI sensors of the LFI sensor array, in particular all LFI sensors of the LFI sensor array, have (in relation to each other) unique emission angles and/or unique emission directions. Thus, user friendliness and/or usability of pairs of smart glasses per se can advantageously be increased. Advantageously, an eye-box of a pair of smart glasses can be significantly enlarged, whereby the system advantageously remains capable of measurement even when a position of the pair of smart glasses, in particular of the LFI sensors of the pair of smart glasses, relative to the eye of the user changes. In particular, the LFI sensor array is designed such that at least a majority of the LFI sensors of the LFI sensor array, preferably all LFI sensors of the LFI sensor array, lie in a common plane. In particular, the LFI sensors of the LFI sensor array are arranged statically/immovably with respect to each other. The fact that the LFI sensor array is statically arranged is in particular understood to mean that the LFI sensor array is arranged immovably in relation to other components of the pair of smart glasses (e.g., glasses lens, glasses frame, glasses temple, apart from any possible fold-in movement of the glasses temple). The term “majority” is in particular understood to mean 60%, preferably 75%, and preferably 90%. The fact that an LFI sensor of the LFI sensor array has a unique emission angle/a unique emission direction is in particular understood to mean that the LFI sensor array, preferably the pair of smart glasses, is free of other LFI sensors with an identical emission angle/an identical emission direction. In particular, the unique emission angle/the unique emission direction occurs only once in the LFI sensor array, preferably in the pair of smart glasses.

According to an example embodiment of the present invention, it is also provided that the oculography device comprises a common lens, which is associated simultaneously with multiple LFI sensors of the LFI sensor array, preferably simultaneously with all LFI sensors of the LFI sensor array, wherein at least a majority of the LFI sensors associated with the common lens, preferably each LFI sensor associated with the common lens, has a different distance and/or a different distance direction with respect to an optical axis of the common lens. Thus, it can be advantageously achieved in a simple and/or inexpensive manner that the LFI sensors of the LFI sensor array have unique emission angles. Thus, an oculography can advantageously be improved. In particular, in this configuration, light from at least a majority of the LFI sensors associated with the common lens, preferably from each of the LFI sensors associated with the common lens, exits the common lens at a different angle.

Alternatively, according to an example embodiment of the present invention, it is provided that the oculography device comprises a common (micro) lens array having a plurality of (micro) lenses, wherein the individual (micro) lenses of the (micro) lens array are each associated with one of the multiple LFI sensors of the LFI sensor array, and wherein in particular at least a majority of the (micro) lenses, preferably all (micro) lenses, have mutually different emission angles. Thus, it can advantageously be achieved in a simple, inexpensive and/or particularly compact manner that the LFI sensors of the LFI sensor array have unique emission angles. Thus, an oculography can advantageously be improved. In particular, the (micro) lenses of the (micro) lens array lie in a common plane, which is preferably parallel to the plane in which the LFI sensors lie. In particular, a microlens has a diameter (perpendicular to the intended transmission direction of the lens) of less than 1 mm, preferably less than 0. 5 mm, and preferably less than 0.1 mm. In particular, exactly one (micro) lens of the (micro) lens array is associated with exactly one LFI sensor of the LFI sensor array in each case. The (micro) lens array may be separate from the LFI sensor array or in the form of a chip-integrated (micro) lens array integrated with the LFI sensor array.

If the LFI sensors of the LFI sensor array have chip-integrated optical units, in particular chip-integrated lenses, a particularly compact design can advantageously be achieved. In addition, the number of parts to be mounted can advantageously be kept small in the manufacture of the pair of smart glasses. The microlenses in particular can be directly integrated with the respective associated LFI sensors.

If in addition the chip-integrated optical unit is formed by a metalens, a particularly space-and/or weight-saving design of the oculography device can advantageously be achieved. In particular, the optical function of the metalens is based on nanostructuring of a surface.

Moreover, according to an example embodiment of the present invention, it is provided that at least two of the LFI sensors of the LFI sensor array, preferably at least three of the LFI sensors of the LFI sensor array, preferably all LFI sensors of the LFI sensor array, are formed by a common multi-cavity laser chip. Thus, production effort can advantageously be reduced, in particular by not having to place a plurality of small LFI sensors, but rather only a single laser chip comprising multiple LFI sensors, relative to an optical system of the pair of smart glasses, e.g., relative to the lens or to the (micro) lens array. In addition, size can advantageously be reduced. It is also possible that the LFI sensor array comprises multiple multi-cavity laser chips each having multiple LFI sensors.

Furthermore, according to an example embodiment of the present invention, it is provided that at least two of the LFI sensors of the LFI sensor array, preferably at least three of the LFI sensors of the LFI sensor array, preferably all LFI sensors of the LFI sensor array, are configured to emit light of different polarization, and that the oculography device comprises a polarization-dependent optical unit which is associated with the LFI sensors emitting differently polarized light. Thus, it can advantageously be achieved in a simple, inexpensive and/or particularly compact manner that the LFI sensors of the LFI sensor array have unique emission angles. In particular, at least in this case the LFI sensors of the LFI sensor array are configured to generate and output differently polarized, preferably differently linearly polarized, light. For example, multiple linearly polarized VCSELs could be correspondingly arranged at a rotational offset to each other in the LFI sensor array. Also possible is the use of VCSELs which each have two laser cavities close to each other having mutually orthogonal polarization. Chips having more than two laser cavities are also possible, the polarization directions of which are, for example, distributed in alternation similarly to a checkerboard pattern. The polarization-dependent optical unit preferably generates different emission angles depending on how the light passing through the polarization-dependent optical unit is polarized. The polarization-dependent optical unit may be in the form of a birefringent crystal, a prism, a polarization-dependently refractive metamaterial, or polarization-dependently refractive liquid crystals.

Furthermore, according to an example embodiment of the present invention, the pair of smart glasses, in particular the pair of AR smart glasses and/or the pair of VR smart glasses/the VR headset, comprising the oculography device is proposed, wherein the pair of smart glasses comprises a hologram unit, which is integrated into a glasses lens of the pair of smart glasses and comprises multiple reflection holograms having different optical functions, which are configured to differently deflect light of different LFI sensors of the LFI sensor array having different angles of incidence. Thus, advantageous designs of pairs of smart glasses comprising the oculography device can be enabled, for example in terms of a construction and/or a design. The light of the LFI sensor array incident from the different angles of incidence is preferably deflected towards the eye of the smart glasses user/an eye positioning region of the pair of smart glasses by the different reflection holograms. The reflection holograms may be arranged in edge regions of the glasses lenses. Thus, good viewing through the glasses lenses can advantageously remain ensured. In a further variant, a material forming the reflection hologram is integrated in the glasses lens over the full area, but only in the edge regions of the glasses lens is said material provided with an optical function causing reflection of the laser beams 41 the LFI sensors. To prevent the perception of boundaries of these active regions, an efficiency of the holographic function can be implemented according to a grayscale gradient in transition regions such that no discrete edge is perceptible.

According to an example embodiment of the present invention, the hologram unit comprises one or more holographic optical elements (HOEs). The HOEs of the hologram unit may be formed together or separate from one another. The reflection holograms of the hologram unit are formed by volume holograms. The volume hologram may be implemented as a continuous optical function or as a piecewise discrete optical function, for example as four quadrants which deflect incident laser beams in different directions to the eye. The reflection holograms of the hologram unit are in particular matched to the wavelength(s) of the LFI sensors. In particular, the reflection holograms of the hologram unit wavelength-selectively reflect only light of the wavelength(s) of the LFI sensors. Light of other wavelengths preferably remains unaffected by the reflection holograms of the hologram unit and passes through them substantially unimpeded.

According to an example embodiment of the present invention, this installation position can become especially advantageous in particular when the LFI sensor array is designed such that at least one LFI sensor thereof emits with a wide angle in different directions. Thus, the laser beam of this LFI sensor preferably hits very different positions of the hologram unit, in particular of the reflection holograms, in the glasses lens. Various optical functions which each deflect this laser beam towards the eye are then imprinted at the different positions of the hologram unit. This makes it possible to irradiate the eye with a laser beam from different directions, so that (in contrast to a fixed installed position of this LFI sensor, e.g., in the lower region of the glasses frame) several projection directions, ideally linearly independent projection directions, of a surface velocity vector of the eye can be produced.

Furthermore, according to an example embodiment of the present invention, a control unit for the oculography device and/or for the pair of smart glasses, which is configured to perform the oculography method, is proposed. Thus, user friendliness and/or usability of pairs of smart glasses per se can advantageously be increased. The term “control unit” is understood in particular to mean a unit comprising at least one control electronics unit. The term “control electronics unit” is understood in particular to mean a unit comprising a processor and comprising a digital memory as well as an operating program stored in the digital memory. In particular, the control unit comprises the operating program with program code comprising commands that, when executed by the processor, cause the control unit to perform the oculography method.

The oculography method according to the present invention, the oculography device according to the present invention, the pair of smart glasses according to the present invention, and the control unit according to the present invention are not intended to be limited to the application and embodiment(s) described above. In order to fulfill a functionality described here, the oculography method according to the present invention, the oculography device according to the present invention, the pair of smart glasses according to the present invention, and the control unit according to the present invention may in particular have a number of individual elements, components, units, and method steps that deviates from a number mentioned here. Moreover, for the value ranges specified in this disclosure, values within the mentioned limits are also to be considered disclosed and usable as desired.

### BRIEF DESCRIPTION OF EXAMPLE EMBODIMENTS

Further advantages result from the following description of the figures. Seven embodiment examples of the present invention are illustrated in the figures. The disclosure herein contain numerous features in combination. A person skilled in the art will expediently also consider the features individually and combine them to form meaningful further combinations, in view of the disclosure herein.

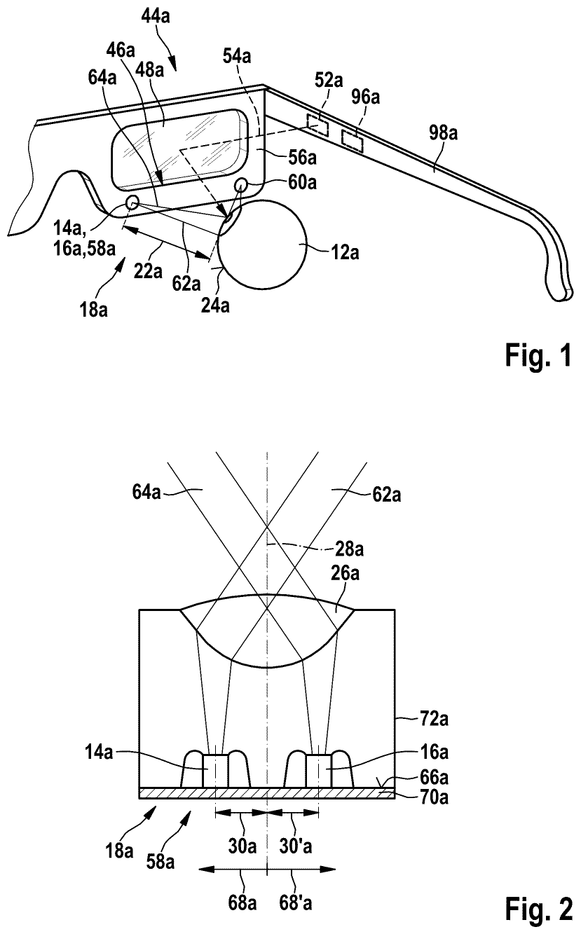

FIG. 1 schematically, by way of example, a pair of smart glasses having an oculography device comprising an LFI sensor array, according to the present invention.

FIG. 2 schematically, by way of example, a sub-array of the LFI sensor array having two LFI sensors, according to the present invention.

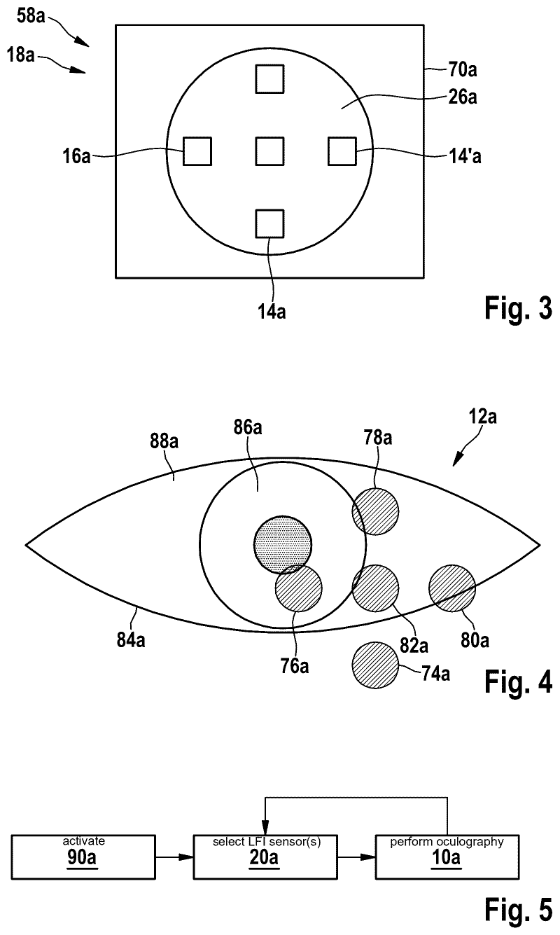

FIG. 3 schematically, a top view of an example embodiment of the sub-array/the LFI sensor array, according to the present invention.

FIG. 4 schematically, an eye of a user of the pair of smart glasses with points of incidence of laser beams of the LFI sensor array, according the present invention.

FIG. 5 shows a schematic flowchart of the oculography method, according to the present invention.

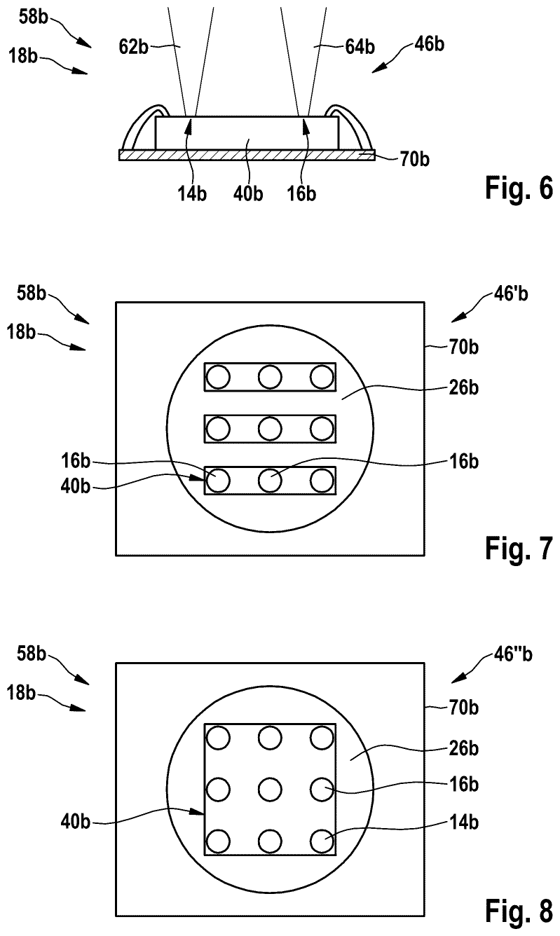

FIG. 6 shows a sub-array of an LFI sensor array or an LFI sensor array of an example embodiment of an alternative oculography device according to the present invention.

FIG. 7 a top view of a first further embodiment of the alternative oculography device, according to the present invention.

FIG. 8 shows a top view of a second further embodiment of the alternative oculography device, according to the present invention.

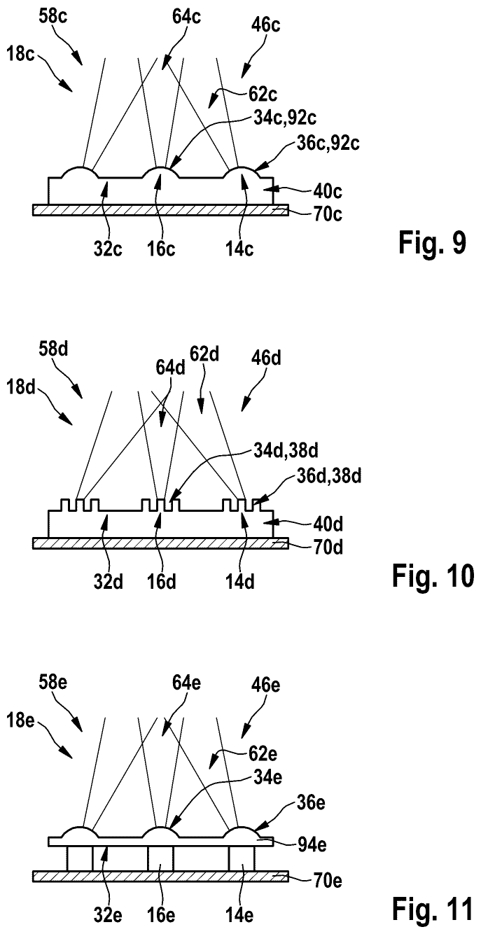

FIG. 9 a schematic side view of a sub-array of an LFI sensor array or an LFI sensor array of a further alternative oculography device, according to an example embodiment of the present invention.

FIG. 10 shows a schematic side view of a sub-array of an LFI sensor array or an LFI sensor array of a second further alternative oculography device, according to an example embodiment of the present invention.

FIG. 11 shows a schematic side view of a sub-array of an LFI sensor array or an LFI sensor array of a third further alternative oculography device, according to an example embodiment of the present invention.

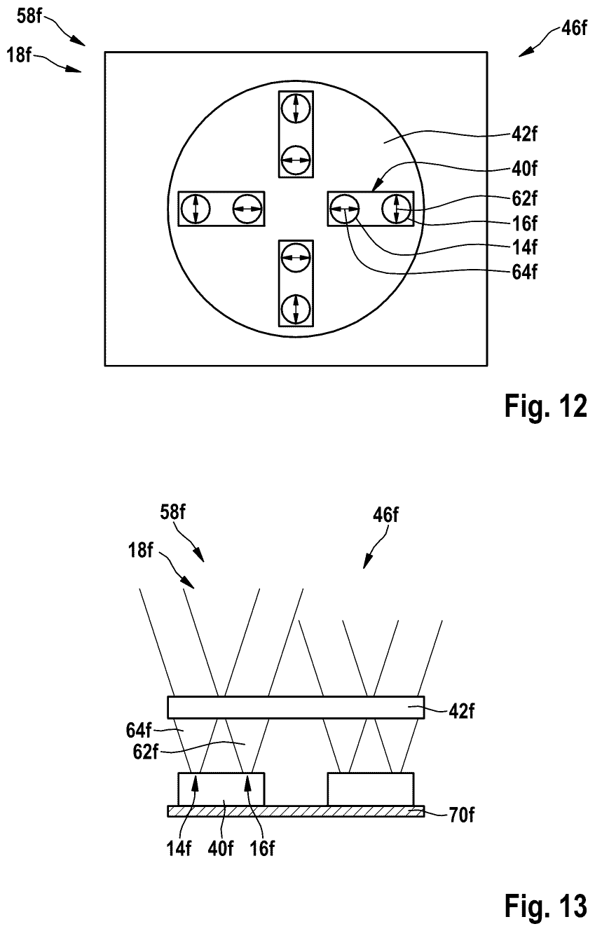

FIG. 12 shows a schematic side view of a sub-array of an LFI Sensor array or an LFI sensor array of a fourth further alternative oculography device, according to an example embodiment of the present invention.

FIG. 13 shows a schematic side view of the sub-array of the LFI sensor array or the LFI sensor array of the fourth further alternative oculography device, according to an example embodiment of the present invention.

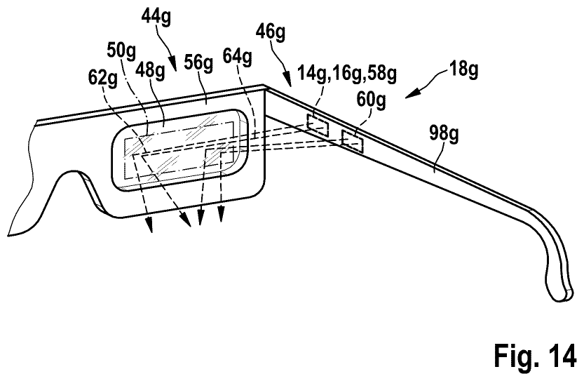

FIG. 14 schematically, an alternative pair of smart glasses with one of the oculography devices described above according to an example embodiment of the present invention.

### DETAILED DESCRIPTION OF EXAMPLE EMBODIMENTS

FIG. 1 schematically shows an exemplary pair of smart glasses 44a. The pair of smart glasses 44a is in the form of a pair of AR smart glasses. The pair of smart glasses 44a comprises a projector unit 52a. The projector unit 52a is configured to output a virtual image by means of a visible laser beam 54a. The pair of smart glasses 44a comprises a glasses lens 48a. In the pair of smart glasses 44a illustrated by way of example in FIG. 1, the glasses lens 48a comprises a holographic optical element configured to deflect the visible laser beam 54a towards an eye 12a of a user. The pair of smart glasses 44a comprises a glasses frame 56a. The glasses frame 56a supports the glasses lens 48a. The pair of smart glasses 44a comprises an oculography device 46a.

The oculography device 46a is configured for performing an oculography method. The oculography device 46a is configured on the basis of the oculography method for tracking a position and/or positioning of the eye 12a within the pair of smart glasses 44a. The oculography device 46a comprises a control unit 96a. The control unit 96a is configured to perform the oculography method. The control unit 96a is, for example, integrated into the pair of smart glasses 44a. Alternatively, the control unit 96a could also be external to the pair of smart glasses 44a (e.g., as a mobile device such as a smartphone, a tablet or a smartwatch, or as a cloud) and in communication connection with the pair of smart glasses 44a. The oculography device 46a comprises a laser feedback interferometry (LFI) sensor array 18a. In the case shown in FIG. 1, the LFI sensor array 18a is integrated into the glasses frame 56a. The LFI sensor array 18a is statically arranged in the pair of smart glasses 44a. In the case shown in FIG. 1, the LFI sensor array also comprises two sub-arrays 58a, 60a. Embodiments with only a single array or more than two arrays are also possible. The LFI sensor array 18a comprises multiple LFI sensors 14a, 16a. Each of the sub-arrays 58a, 60a comprises multiple LFI sensors 14a, 16a. The LFI sensors provided with reference signs 14a and 16a here are, by way of example, associated with a first sub-array 58a of the sub-arrays 58a, 60a. The LFI sensors 14a, 16a are each configured at least for ascertaining and/or tracking at least one eye movement of the eye 12a. Each of the LFI sensors 14a, 16a of the LFI sensor array 18a is provided for outputting a laser beam 62a, 64a. The laser beams 62a, 64a of LFI sensors 14a, 16a are invisible to the eye 12a. The laser beams 62a, 64a of the LFI sensors 14a, 16a are infrared laser beams (e. g., having a wavelength of about 850 nm or about 940 nm). The laser beams 62a, 64a have an intensity of less than 1 mW (optical output power). The laser beams 62a, 64a are safe for eyes. The laser beams 62a, 64a may be collimated or may be focused to an optical path length 22a between the associated LFI sensor 14a, 16a and a scattering surface 24a, e.g., of the eye 12a. The LFI sensors 14a, 16a of the LFI sensor array 18a each have unique emission angles. The LFI sensors 14a, 16a of the LFI sensor array 18a each have unique emission directions. The emission angles and/or emission directions of the laser beams 62a, 64a of the LFI sensors 14a, 16a are each unique.

The laser beams 62a, 64a of the LFI sensors 14a, 16a hit a face surface of the user of the pair of smart glasses 44a or an eye surface of the eye 12a of the user of the pair of smart glasses 44a and are scattered there. Thus, the eye surface and/or the face surface each form a scattering surface 24a. The scattering at the scattering surface 24a scatters a portion of the respective laser beam 62a, 64a back into the respective LFI sensor 14a, 16a, in particular into a laser cavity of the respective LFI sensor 14a, 16a, thereby generating a sensor signal useful for the oculography method.

FIG. 2 schematically shows an exemplary sub-array 58a of the LFI sensor array 18a having two LFI sensors 14a, 16a. The LFI sensor array 18a could also be formed entirely by that shown in FIG. 2. The oculography device 46a has a common lens 26a. The common lens 26a is simultaneously associated with multiple LFI sensors 14a, 16a of the LFI sensor array 18a. The common lens 26a is simultaneously associated with the two sensors 14a, 16a shown in FIG. 2. The common lens 26a has an optical axis 28a.

The optical axis 28a forms an axis of symmetry of the rotationally symmetric optical system of lens 26a. The two LFI sensors 14a, 16a with which the common lens 26a is associated have a different distance 30a, 30′a from the optical axis 28a of the common lens 26a. The two LFI sensors 14a, 16a with which the common lens 26a is associated have a different distance direction 68a, 68′a with respect to the optical axis 28a of the common lens 26a. The distances 30a, 30′a extend in a plane parallel to an array plane 66a of the LFI sensor array 18a. The distance directions 68a, 68′a extend in a plane parallel to an array plane 66a of the LFI sensor array 18a. A first LFI sensor 14a of the LFI sensors 14a, 16a has a first distance 30a from the optical axis 28a, the first distance being perpendicular to optical axis 28a. The first LFI sensor 14a of the LFI sensors 14a, 16a has a first distance direction 68a pointing perpendicularly towards the optical axis 28a. A second LFI sensor 16a of the LFI sensors 14a, 16a has a second distance 30′a from the optical axis 28a, the second distance being perpendicular to optical axis 28a. The second LFI sensor 16a of the LFI sensors 14a, 16a has a second distance direction 68a pointing perpendicularly towards the optical axis 28a. The first distance 30a and the second distance 30′a are different. The first distance direction 68a and the second distance direction 68′a point in different directions.

The sub-array 58a/the LFI sensor array 18a comprises a carrier substrate 70a. The carrier substrate 70a is designed as an electrical conducting track/a submount. The sub-array 58a/the LFI sensor array 18a comprises a housing 72a. The common lens 26a is set in the housing 72a. The LFI sensors 14a, 16a are positioned relative to the common lens 26a such that their laser beams 62a, 64a both pass through the common lens 26a but, due to their different angles of incidence on a surface of the common lens 26a, produce significantly different exit angles.

FIG. 3 schematically shows a top view of an embodiment example of the sub-array 58a/the LFI sensor array 18a with five LFI sensors 14a, 16a disposed on a common carrier substrate 70a, under a single common lens 26a. In this embodiment example, the centrally arranged LFI sensor 14′a radiates perpendicularly to the carrier substrate 70a, while the eccentrically arranged LFI sensors 14a, 16a emit at different exit angles to the optical axis 28a. Because of the distance (optical path length 22a) between the installation location of the LFI sensors 14a, 14′a, 16a (e. g., in the glasses frame 56a) and the eye 12a, the laser beams 62a, 64a of the sub-array 58a/the LFI sensor array 18a are incident on the eye 12a at different locations due to the different exit angles of said laser beams.

This is shown schematically in FIG. 4, which depicts the eye 12a and points of incidence 74a, 76a, 78a, 80a, 82a of the laser beams 62a, 64a of the sub-array 58a/the LFI sensor array 18a on and around the eye 12a. A first point of incidence 74a is on an eyelid 84a of the eye 12a. A second point of incidence 76a is on an iris 86a of the eye 12a. A third point of incidence 78a, a fourth point of incidence 80a and a fifth point of incidence 82a are predominantly on a sclera 88a of the eye 12a.

FIG. 5 shows a schematic flowchart of the oculography method. In at least one activation step 90a, the pair of smart glasses 44a and/or the oculography device 46a, in particular the LFI sensor array 18a, is activated. In at least one selection step 20a, a selection of at least one LFI sensor 14a, 14′a, 16a of the LFI sensor array 18a provided for performing an oculography step 10a following the selection step 20a in time is made on the basis of a parameter comparison of test measurements of multiple LFI sensors 14a, 14′a, 16a of the LFI sensor array 18a. For this purpose, the LFI sensors 14a, 14′a, 16a are operated in succession or simultaneously to perform at least one test measurement each. The test measurements are each velocity test measurements. In the velocity test measurements, respective raw velocities of movements of the scattering surfaces 24a are ascertained at the points of incidence 74a, 76a, 78a, 80a, 82a of the individual laser beams 62a, 64a. The observed measurement signal of the velocity test measurements provides an indication as to whether the associated LFI sensor 14a, 14′a, 16a currently hits the eye 12a well or not. In the example of FIG. 4, no velocity would be observed at the first point of incidence 74a, since it lies on the lower eyelid 84a of the eye 12a and thus no velocity is detectable. At the fourth point of incidence 80a, in the example shown, the associated laser beam 62a, 64a could strike the scattering surface 24a, in particular an eyeball surface of the eye 12a, at a right angle such that a surface velocity component in the laser beam direction is zero. The LFI sensor 14a, 14′a, 16a associated with the fourth point of incidence 80a would thus likewise be unsuitable. Thus, in the example shown in FIG. 4, one of the LFI sensors 14a, 14′a, 16a of the LFI sensor array 18a whose laser beam 62a, 64a generates one of the points of incidence 76a, 78a, 82a would generate the largest measurement signal. The LFI sensor 14a, 14′a, 16a with the highest velocity ascertained in the selection step 20a is selected for performing the following oculography step 10a. In the oculography step 10a, at least one eye movement of the eye 12a is ascertained, in particular tracked, by one or more of the LFI sensors 14a, 14′a, 16a which delivered the highest velocity in the selection step 20a.

The selection step 20a is repeated following a first performance of the oculography step 10a if a time interval, in particular definable time interval, has passed, if it is ascertained that a minimum measurement data quality is not met when the oculography step 10a is performed, and/or if a substantial change in the optical path length 22a ascertained by the currently selected LFI sensor 14a, 14′a, 16a is ascertained.

FIGS. 6 to 14 show further embodiment examples of the present invention. The following descriptions and the drawings are substantially limited to the differences between the embodiment examples, wherein reference can also be made in principle to the drawings and/or the description of the other embodiment examples, in particular FIGS. 1 to 5, with respect to identically described components, in particular with respect to components having the same reference numerals. To differentiate between the embodiment examples, the letter a is placed after the reference numerals of the embodiment example in FIGS. 1 to 5. In the embodiment examples of FIGS. 6 to 14, the letter a is replaced by the letters b to g.

FIG. 6 shows a sub-array 58b of an LFI sensor array 18b or an LFI sensor array 18b of an alternative oculography device 46b. The sub-array 58b or the LFI sensor array 18b comprises, by way of example, at least two LFI sensors 14b, 16b. The at least two LFI sensors 14b, 16b are formed by a common multi-cavity laser chip 40b.

FIG. 7 shows a top view of a further embodiment of the alternative oculography device 46′b. In this view it can be seen that, in this embodiment, even three LFI sensors 14b, 16b are arranged in a common multi-cavity laser chip 40b. The sub-array 58b or the LFI sensor array 18b also comprises multiple multi-cavity laser chips 40b which are collectively disposed under a common lens 26b of the alternative oculography device 46′b. In a second further embodiment of the alternative oculography device 46′b shown in FIG. 8, all LFI sensors 14b, 16b associated with the common lens 26b are combined in a common multi-cavity laser chip 40b. In this case, the multi-cavity laser chip 40b comprises nine LFI sensors 14b, 16b.

FIG. 9 shows a schematic side view of a sub-array 58c of an LFI sensor array 18c or an LFI sensor array 18c of a further alternative oculography device 46c. The LFI sensor array 18c comprises multiple LFI sensors 14c, 16c. This embodiment addresses the problem that, when a common lens 26a, 26b is used, relatively large distances between the laser cavities of the LFI sensors 14c, 16c are required for large angular differences between the exiting laser beams 62c, 64c. The associated large chip area may influence the efficiency of semiconductor manufacturing, since it could be possible to produce many more laser cavities on the same area. To be able to place the laser cavities of the LFI sensors 14c, 16c of LFI sensor array 18c as close to one another as possible, the LFI sensors 14c, 16c of LFI sensor array 18c of the further alternative oculography device 46c have chip-integrated optical units 92c. The chip-integrated optical units 92c of the LFI sensors 14c, 16c of the LFI sensor array 18c of the further alternative oculography device 46c form chip-integrated lenses. The emission angles of each laser cavity are modified separately by the chip-integrated optical units 92c.

In the embodiment shown by way of example in FIG. 9, respective rear sides of the LFI sensors 14c, 16c have been provided with chip-integrated microlens arrays 32c using grayscale lithography. However, in general it is irrelevant to the present invention whether the LFI sensors 14c, 16c are configured to be front-emitting or rear-emitting. The further alternative oculography device 46c thus comprises a (common) microlens array 32c. The microlens array 32c comprises a plurality of microlenses 34c, 36c. Each of the individual microlenses 34c, 36c of the microlens array 32c is associated with a single one of the LFI sensors 14c, 16c of the LFI sensor array 18c. The microlenses 34c, 36c of the microlens array 32c have mutually different emission angles. It is possible that, after the emission angle has been set by the microlens array 32c, a further lens (not shown) for setting a collimation state of the deflected laser beams 62c, 64c is arranged downstream.

FIG. 10 shows a schematic side view of a sub-array 58d of an LFI sensor array 18d or an LFI sensor array 18d of a second further alternative oculography device 46d. The LFI sensor array 18d comprises multiple LFI sensors 14d, 16d. The second further alternative oculography device 46d comprises a (common) microlens array 32d. The microlens array 32d comprises a plurality of microlenses 34d, 36d. Each of the individual microlenses 34d, 36d of the microlens array 32d is associated with a single one of the LFI sensors 14d, 16d of the LFI sensor array 18d. The microlenses 34d, 36d of the microlens array 32d are in the form of chip-integrated optical units 92d. The microlenses 34d, 36d of the microlens array 32d are in the form of metalenses 38d. The chip-integrated optical units 92d are formed by metalenses 38d. The metalens 38d is, for example, formed by a binary pillar structure. Alternative conventional metalens shapes that make laser beam deflection possible are also possible.

FIG. 11 shows a schematic side view of a sub-array 58e of an LFI sensor array 18e or an LFI sensor array 18e of a third further alternative oculography device 46e. The LFI sensor array 18e comprises multiple LFI sensors 14e, 16e. The third further alternative oculography device 46e comprises a (common) microlens array 32e. The microlens array 32e comprises a plurality of microlenses 34e, 36e. Each of the individual microlenses 34e, 36e of the microlens array 32e is each associated with a single one of the LFI sensors 14e, 16e of the LFI sensor array 18e. The microlenses 34e, 36e of the microlens array 32e are separate from the LFI sensors 14e, 16e. The microlens array 32e is in the form of a separate lens plate 94e. The separate lens plate 94e comprises the plurality of microlenses 34e, 36e. The microlens array 32e is manufactured separately from the LFI sensors 14e, 16e. The separate lens plate 94e is combined with the LFI sensors 14e, 16e by a wafer-to-wafer process.

FIG. 12 shows a schematic top view of a sub-array 58f of an LFI sensor array 18f or an LFI sensor array 18f of a fourth further alternative oculography device 46f. The LFI sensor array 18f comprises multiple LFI sensors 14f, 16f. At least two of the LFI sensors 14f, 16f are combined in each of common multi-cavity laser chips 40f. The LFI sensors 14f, 16f of a common multi-cavity laser chip 40f emit mutually orthogonally polarized laser beams 62f, 64f (indicated by double arrows).

Thus, two of the LFI sensors 14f, 16f of the LFI sensor array 18f are configured to emit light of different polarization. The fourth further alternative oculography device 46f has a polarization-dependent optical unit 42f. The polarization-dependent optical unit 42f is provided for polarization-dependent deflection of incident light. The polarization-dependent optical unit 42f is associated with the LFI sensors 14f, 16f emitting differently polarized light. The respective laser beams 62f, 64f approximately uniformly enter the polarization-dependent optical unit 42f but exit the polarization-dependent optical unit 42f at substantially different exit angles to each other. FIG. 13 shows a schematic side view of the sub-array 58f of LFI sensor array 18f or the LFI sensor array 18f of the fourth further alternative oculography device 46f with, by way of example, two double-cavity chips each radiating two laser beams 62f, 64f of different polarization, which are then differently deflected by polarization-dependent optical unit 42f.

The optical arrangements (individually or in combination) shown in FIGS. 1 to 13, make nearly any desired beam manipulations possible, in particular because a combination of polarization state, position of the laser chip with respect to the lens and region of the lens is possible. Thus, angular shift and parallel shift of the laser beams 62a-f, 64a-f can be individually set in order to set a particularly advantageous laser spot distribution on the user's eye region.

FIG. 14 schematically shows alternative pair of smart glasses 44g having one of the oculography devices 46a, 46b, 46c, 46d, 46e, 46f described above. The alternative pair of smart glasses 44g has a glasses lens 48g. A hologram unit 50g is integrated into the glasses lens 48g. The hologram unit 50g has a plurality of reflection holograms having different optical functions. The reflection holograms of the hologram unit 50g having the different optical functions are configured to differently deflect light of different LFI sensors 14g, 16g of an LFI sensor array 18g of the pair of smart glasses 44g having different angles of incidence. The LFI sensor array 18g is integrated into a glasses temple 98g of the pair of smart glasses 44g. The reflection holograms of the hologram unit 50g may be arranged only in a region of an edge of the glasses lens 48g or may extend over a large part of the glasses lens 48g. In the latter case, an efficiency of the holographic function of the reflection hologram of the hologram unit 50g could increase from a center of the glasses lens 48g towards an edge of the glasses lens 48g.

1-14. (canceled)

15. A smart-glasses oculography method for pairs of augmented reality smart glasses and/or for pairs of virtual reality (VR) smart glasses/VR headsets, comprising:
in at least one oculography step, tracking at least one eye movement of an eye of a smart-glasses user, by a subset of laser feedback interferometry (LFI) sensors of an LFI sensor array which is associated with the eye; and
in at least one selection step which precedes the oculography step in time, making a selection of at least one LFI sensor provided for performing the oculography step, which follows in time, based on a parameter comparison of test measurements of multiple LFI sensors of the LFI sensor array, the selected at least one LFI sensor forming the subset.

16. The oculography method according to claim 15, wherein, in the selection step, the selection of the at least one LFI sensor of the LFI sensor array provided for performing the oculography step is made based on a parameter comparison of eye movement velocity test measures with the multiple LFI sensors of the LFI sensor array.

17. The oculography method according to claim 16, wherein at least the LFI sensor having a highest velocity ascertained in the selection step is selected for performing the oculography step.

18. The oculography method according to claim 16, wherein the selection step is repeated when a definable time interval has passed, when it is ascertained that a minimum measurement data quality is not met when the oculography step is performed, and/or when a substantial change in an optical path length ascertained by a currently selected LFI sensor between LFI sensor and scattering surface including the eye of the smart glasses user is ascertained.

19. An oculography device for performing an oculography method, comprising:
at least one laser feedback interferometry (LFI) sensor array which includes multiple LFI sensors each configured to track at least one eye movement of an eye of a smart glasses user, at least a majority of all of the LFI sensors of the LFI sensor array have unique emission angles and/or unique emission directions;
wherein the oculography device is configured to perform:
in at least one oculography step, tracking the at least one eye movement of the eye of a smart-glasses user, by a subset of the LFI sensors of the LFI sensor array which is associated with the eye; and
in at least one selection step which precedes the oculography step in time, making a selection of at least one LFI sensor provided for performing the oculography step, which follows in time, based on a parameter comparison of test measurements of multiple LFI sensors of the LFI sensor array, the selected at least one LFI sensor forming the subset.

20. The oculography device according to claim 19, further comprising:
a common lens, which is associated simultaneously with multiple LFI sensors of the LFI sensor array, wherein at least a majority of the LFI sensors associated with the common lens has a different distance and/or a different distance direction with respect to an optical axis of the common lens.

21. The oculography device according to claim 19, further comprising:
a common microlens array having a plurality of microlenses, wherein each of the microlenses of the microlens array is associated with one of the multiple LFI sensors of the LFI sensor array, and wherein at least a majority of the microlenses have mutually different emission angles.

22. The oculography device according to claim 19, wherein the LFI sensors of the LFI sensor array include chip-integrated optical units.

23. The oculography device according to claim 22, wherein each of the chip-integrated optical units is a metalens.

24. The oculography device according to claim 19, wherein at least two of the LFI sensors of the LFI sensor array are formed by a common multi-cavity laser chip.

25. The oculography device according to claim 19, wherein at least two of the LFI sensors of the LFI sensor array are configured to emit light of different polarization, and wherein a polarization-dependent optical unit which is associated with the LFI sensors emits differently polarized light.

26. A pair of smart glasses, comprising:
an oculography device including:
at least one laser feedback interferometry (LFI) sensor array which includes multiple LFI sensors each configured to track at least one eye movement of an eye of a smart glasses user, at least a majority of all of the LFI sensors of the LFI sensor array have unique emission angles and/or unique emission directions;
wherein the oculography device is configured to perform:
in at least one oculography step, tracking the at least one eye movement of the eye of a smart-glasses user, by a subset of the LFI sensors of the LFI sensor array which is associated with the eye; and
in at least one selection step which precedes the oculography step in time, making a selection of at least one LFI sensor provided for performing the oculography step, which follows in time, based on a parameter comparison of test measurements of multiple LFI sensors of the LFI sensor array, the selected at least one LFI sensor forming the subset.

27. The smart glasses according to claim 26, wherein a hologram unit is integrated into a glasses lens of the pair of smart glasses and includes a plurality of reflection holograms having different optical functions, which are configured to differently deflect light of different LFI sensors of the LFI sensor array having different angles of incidence.

28. A control unit for an oculography device for a pair of smart glasses configured to perform an oculography method, comprising:
in at least one oculography step, tracking at least one eye movement of an eye of a smart-glasses user, by a subset of laser feedback interferometry (LFI) sensors of an LFI sensor array which is associated with the eye; and
in at least one selection step which precedes the oculography step in time, making a selection of at least one LFI sensor provided for performing the oculography step, which follows in time, based on a parameter comparison of test measurements of multiple LFI sensors of the LFI sensor array, the selected at least one LFI sensor forming the subset.
